(19) United States
(12) Patent Application Publication
(10) Pub. No.: US 2022/0373563 A1
(43) Pub. Date: Nov. 24, 2022

(54) MACHINE LEARNING-BASED AUTISM SPECTRUM DISORDER DIAGNOSIS METHOD AND DEVICE USING METABOLITE AS MARKER

(71) Applicant: PEKING UNION MEDICAL COLLEGE HOSPITAL, Beijing (CN)

(72) Inventor: Xin YOU, Beijing (CN)

(21) Appl. No.: 17/619,327

(22) PCT Filed: Apr. 23, 2019
(86) PCT No.: PCT/CN2019/083944

§ 371 Date: Dec. 15, 2021

(51) Int. Cl. G01N 33/68; G01N 33/487; G16H 50/20

(52) U.S. Cl. CPC G01N 33/6896; G01N 33/487; G16H 50/20; G01N 2800/28

(57) ABSTRACT

Provided are a machine learning-based autism spectrum disorder (ASD) diagnosis method and device using a metabolite as a marker. The method comprises: measuring the content of at least one marker in a sample of a subject and comparing same with the content of the corresponding marker in a healthy control, or using an algorithm constructed by machine learning to process the content of the marker. Particularly, the marker is a metabolite in human urine. The device comprises: an accommodation space, configured to place the sample of the subject; a testing unit, configured to test the marker in the sample to obtain the content of the marker; and a calculation and determination unit, configured to perform calculation on the basis of the content of the marker according to a predetermined algorithm to obtain an indication of whether the subject suffers from ASD. According to the present application, the change pattern of a metabolite in urine is mined by means of a machine learning algorithm to provide diagnoses for children suffering from ASD. The device based on a predetermined algorithm provided by the present application can provide a new strategy for diagnosis of ASD.

### TECHNICAL FIELD

The present invention relates to methods and devices for diagnosing autism spectrum disorder.

### BACKGROUND

Autism spectrum disorder (ASD) is a neurodevelopmental disorder, characterized as communication disorder and social disorder, and repetitive and stereotyped behaviors are the main manifestations. At present, the etiology of autism spectrum disorder is not clear, and it is generally considered to be caused by a combination of genetic and environmental factors in the first few critical developmental years. At present, there is a lack of biomarkers for the diagnosis of autism spectrum disorder. and a lack of effective detection methods.

The diagnosis of autism spectrum disorder relies on professional psychiatrists and psychologists' assessment with the use of behavioral methods. The commonly used scales are DSM-4 (Diagnostic and Statistical Manual of Mental Disorders, Fourth Edition) and DSM-5 of the APA (American Psychiatric Association). The usual diagnostic methods focus on behavioral characteristics, which makes the diagnosis of patients under three years old very difficult. Moreover, ASD is highly heterogeneous, and the behavioral manifestations vary from person to person, making the diagnosis of autism spectrum disorder very difficult. The early diagnosis and early treatment of autism spectrum disorder are critical to the prognosis, and the delay in diagnosis will cause the loss of the best opportunity for the treatment and intervention for child patients.

### SUMMARY

The present invention provides a method for constructing a mathematical model for diagnosing autism spectrum disorder, including

(1) Providing samples of a first group of subjects and a second group of subjects;

(2) Detecting the content of at least one marker in the samples of the first group of subjects and the samples of the second group of subjects, respectively, to obtain data;

(3) Using machine learning algorithms to process the data to obtain a mathematical model for diagnosing autism spectrum disorder,

and wherein the first group of subjects includes subjects diagnosed with autism spectrum disorder, and the second group of subjects includes healthy subjects.

In one or more embodiments, in the processing step of using the machine learning algorithm, the content of at least one marker obtained from the sample of the first group of subjects is divided into a first data set and a second data set, the content of at least one marker obtained from the samples of the second group of subjects is divided into a third data set and a fourth data set, wherein the first and the third data sets are grouped into a training set, and the second and the fourth data sets are grouped into a test set, and the training set and the test set are used for processing by the machine learning algorithm.

In one or more embodiments, the first group of subjects includes pediatric patients diagnosed with autism spectrum disorder.

In one or more embodiments, the machine learning algorithm includes at least one of a Partial Least Squares-Discriminant Analysis algorithm (PLSDA), a Support Vector Machine algorithm (SVM), or an eXtreme Gradient Boosting (XGBoost) algorithm.

In one or more embodiments, the machine learning algorithm is an eXtreme Gradient Boosting algorithm.

In one or more embodiments, the samples of the first group of subjects and the samples of the second group of subjects include at least one of urine, blood, phlegm, nasopharyngeal secretions, body fluids, or feces.

In one or more embodiments, the samples from the first group of subjects and the second group of subjects are urine.

In one or more embodiments, the marker is selected from metabolites.

In one or more embodiments, the marker includes at least one of Phenylactic acid, 3-Hydroxy-3-Methylglutaric acid, Phosphoric acid, Fumaric acid, 3-Oxoglutaric acid, Aconitic acid, N-Acetylcysteine, Malonic acid, Tricarboxylic acid, Glycolic acid, Creatinine, Malic acid, Oxalic acid, Tartaric acid, Pyruvic acid, 4-Cresol, Carboxycitric acid, 3-Hydroxyglutaric acid, 2-Hydroxybutyric acid, or 2-Oxoglutaric acid.

In one or more embodiments, the marker is selected from the group consisting of Phenylactic acid, 3-Hydroxy-3-Methylglutaric acid, Phosphoric acid, Fumaric acid, 3-Oxoglutaric acid, Aconitic acid, N-Acetylcysteine, Malonic acid, Tricarboxylic acid, Glycolic acid, Creatinine, Malic acid, Oxalic acid, Tartaric acid, Pyruvic acid, 4-Cresol, Carboxycitric acid, 3-Hydroxyglutaric acid, 2-Hydroxybutyric acid, and 2-Oxoglutaric acid.

In one or more embodiments, the marker includes at least one of Phenylactic acid, Aconitic acid, Phosphoric acid, 3-Oxoglutaric acid or Carboxycitric acid.

In one or more embodiments, the marker is selected from the group consisting of Phenylactic acid, Aconitic acid, Phosphoric acid, 3-Oxoglutaric acid, and Carboxycitric acid.

In one or more embodiments, the detection in step (2) is achieved by gas chromatography.

In one or more embodiments, the detection step (2) is achieved by a combination of gas chromatography and mass spectrometry.

The present invention provides a method for diagnosing autism spectrum disorder, including:

(a) Determining the content of at least one marker in a sample of a subject to obtain data; and

(b) Using a mathematical model for diagnosing autism spectrum disorder described in the present invention to process the data.

The present invention provides a method for diagnosing autism spectrum disorder, including:

(i) Determining the content of at least one marker in a sample of a subject to obtain data, and

(Ii) Processing the data.

In one or more embodiments, the method for diagnosing autism spectrum disorder disclosed in the present invention further includes, prior to step (ii), determining the content of at least one marker in a sample of healthy individuals to obtain data, and in step (ii) the processing includes comparing the data of the content of at least one marker in the sample of the subject with the data of the content of the corresponding marker in the sample of healthy individuals.

In one or more embodiments, in the method for diagnosing autism spectrum disorder of disclosed in the present invention, the processing in step (ii) includes processing the data using the mathematical model for diagnosing autism spectrum disorder disclosed in the present invention.

In one or more embodiments, the subject is a human.

In one or more embodiments, the subject is a child.

In one or more embodiments, the subject is a child 3 years old or younger.

In one or more embodiments, the sample of the subject includes at least one of urine, blood, sputum, nasopharyngeal secretions, body fluids, or feces.

In one or more embodiments, the sample of the subject is urine.

In one or more embodiments, the marker is selected from metabolites.

In one or more embodiments, the marker includes at least one of Phenylactic acid, 3-Hydroxy-3-Methylglutaric acid, Phosphoric acid, Fumaric acid, 3-Oxoglutaric acid, Aconitic acid, N-Acetylcysteine, Malonic acid, Tricarboxylic acid, Glycolic acid, Creatinine, Malic acid, Oxalic acid, Tartaric acid, Pyruvic acid, 4-Cresol, Carboxycitric acid, 3-Hydroxyglutaric acid, 2-Hydroxybutyric acid, or 2-Oxoglutaric acid.

In one or more embodiments, the marker includes at least one, two, three, four, five, six, seven, eight, nine, ten, eleven, twelve, thirteen, fourteen, fifteen, sixteen, seventeen, eighteen, nineteen, or all of Phenylactic acid, 3-Hydroxy-3-Methylglutaric acid, Phosphoric acid, Fumaric acid, 3-Oxoglutaric acid, Aconitic acid, N-Acetylcysteine, Malonic acid, Tricarboxylic acid, Glycolic acid, Creatinine, Malic acid, Oxalic acid, Tartaric acid, Pyruvic acid, 4-Cresol, Carboxycitric acid, 3-Hydroxyglutaric acid, 2-Hydroxybutyric acid, or 2-Oxoglutaric acid.

In one or more embodiments, the marker is selected from the group consisting of Phenylactic acid, 3-Hydroxy-3-Methylglutaric acid, Phosphoric acid, Fumaric acid, 3-Oxoglutaric acid, Aconitic acid, N-Acetylcysteine, Malonic acid, Tricarboxylic acid, Glycolic acid, Creatinine, Malic acid, Oxalic acid, Tartaric acid, Pyruvic acid, 4-Cresol, Carboxycitric acid, 3-Hydroxyglutaric acid, 2-Hydroxybutyric acid, and 2-Oxoglutaric acid.

In one or more embodiments, the marker includes at least one of Phenylactic acid, Aconitic acid, Phosphoric acid, 3-Oxoglutaric, and Carboxycitric acid.

In one or more embodiments, the marker comprising Phenylactic acid, Aconitic acid, Phosphoric acid, 3-Oxoglutaric, and carboxycitric acid.

In one or more embodiments, the determination in step (a) or (i) is achieved by gas chromatography.

In one or more embodiments, the determination in step (a) or (i) is achieved by a combination of gas chromatography and mass spectrometry.

In one or more embodiments, the autism is selected from Rett syndrome, childhood disintegration, Asperger's syndrome, and unspecified generalized developmental disorder.

The present invention provides a device for diagnosing autism spectrum disorder, including

an accommodating space configured to place a sample of a subject;

a detection unit configured to detect a marker of the sample to obtain the content of the marker; and

a calculation and determination unit configured to calculate the content of the marker according to a predetermined algorithm to obtain an indication of whether the subject suffers from autism spectrum disorder.

In the device described above, the predetermined algorithm is at least one of PLSDA, SVM, and XGBoost.

In the above-mentioned device, the detection unit is selected from a gas chromatography detection device and a liquid chromatography device.

In the above-mentioned device, the detection unit includes a gas chromatography detection device and a mass spectrometry detection device.

The device as described above, wherein the sample includes at least one of urine, blood, sputum, nasopharyngeal secretions, body fluids, or feces.

The device as described above, wherein the marker includes at least one of Phenylactic acid, 3-Hydroxy-3-Methylglutaric acid, Phosphoric acid, Fumaric acid, 3-Oxoglutaric acid, Aconitic acid, N-Acetylcysteine, Malonic acid, Tricarboxylic acid, Glycolic acid, Creatinine, Malic acid, Oxalic acid, Tartaric acid, Pyruvic acid, 4-Cresol, Carboxycitric acid, 3-Hydroxyglutaric acid, 2-Hydroxybutyric acid, or 2-Oxoglutaric acid.

In the device as described above, the marker is selected from a group consisting of Phenylactic acid, 3-Hydroxy-3-Methylglutaric acid, Phosphoric acid, Fumaric acid, 3-Oxoglutaric acid, Aconitic acid, N-Acetylcysteine, Malonic acid, Tricarboxylic acid, Glycolic acid, Creatinine, Malic acid, Oxalic acid, Tartaric acid, Pyruvic acid, 4-Cresol, Carboxycitric acid, 3-Hydroxyglutaric acid, 2-Hydroxybutyric acid, and 2-Oxoglutaric acid.

In the device as described above, the marker includes at least one of Phenylactic acid, Aconitic acid, Phosphoric acid, 3-Oxoglutaric acid, or Carboxycitric acid.

In the device as described above, the marker comprising Phenylactic acid, Aconitic acid, Phosphoric acid, 3-Oxoglutaric acid and Carboxycitric acid.

The beneficial effects of the present invention include but are not limited to the following:

(1) The present invention applies machine learning algorithms to disease marker screening and disease diagnosis mathematical model establishment. In particular, Partial Least Square Discriminant Analysis, Support Vector Machine and XGBoost algorithm were used to screen out the 20 most weighted markers, and a highly effective diagnostic model was established using XGBoost.

(2) The present invention uses urine as a sample. The urine collection method is simple and easy to implement, and the urine collection is a non-invasive process, and has a high operability in the clinic. These are conducive to the diagnosis of autistic patients.

(3) The present invention has successfully established a diagnosis model for autism based on 20 or more metabolites. And using the mathematical model of the present invention to process the sample parameters greatly improved the specificity, sensitivity, and practicability of the diagnosis.

(4) Chromatography-mass spectrometry can quickly detect 20 or more metabolites at once. This method is fast and relatively cheap.

(5) The mathematical model and device of the present invention can be used for early diagnosis of autism spectrum disorder. It overcomes the bottleneck in autism spectrum disorder disease diagnosis, i.e., diagnosing without objective indicators. It solves the technical problem to diagnose children with autism aged 3 years or younger.

(6) A comprehensive study of the metabolites of patients with autism spectrum disorder will also provide clues for the study of the biological phenotype and disease pathogenesis of autism spectrum disorder.

### BRIEF DESCRIPTION OF THE DRAWINGS

In order to explain the technical solutions of the embodiments of the present invention more clearly, the following will briefly describe the drawings used in the embodiments. It should be understood that the following drawings are only certain embodiments of the present invention, therefore, it should be regarded as a limitation of the scope of the present invention. Those of ordinary skill in the art, without additional inventive work, can obtain other related drawings based on these drawings.

FIG. 1 is a schematic diagram showing an apparatus for diagnosing autism spectrum disorder according to an embodiment of the present invention.

FIG. 2: a) ROC based on the final model of an independent test set of all 76 metabolites. b) ROC based on the final model of an independent test set of the first 20 metabolites. c) ROC based on the final model of an independent test set of the first 5 metabolites. d) AUR curve for selected metabolites. The first 20 metabolites represent the best set of possible ASD biomarkers, and adding more other metabolites will reduce the AUR of SVM and PLSDA. The AUR of the XGBoost algorithm reaches a plateau after including 20 metabolites, and no longer increases.

FIG. 3 shows the heat map analysis of GC/MS metabolomics. The rows and columns represent metabolites and samples, respectively. The decrease and increase of metabolites are shown in blue and red, respectively. If the level of metabolites in the same cluster in children with autism spectrum disorder is abnormally high or low, an intuitive red or blue color block will appear in the graph.

### DETAILED DESCRIPTION

In order to make the objectives, technical solutions, and advantages of the embodiments of the present invention clearer, the technical solutions in the embodiments of the present invention will be described clearly and completely below. If the specific conditions are not specified in the examples, it shall be carried out in accordance with the conventional conditions, or the conditions recommended by the manufacturer. The reagents or instruments used without specifying the manufacturers should be understood to be conventional products that can be purchased on the market.

### Definitions and General Techniques

Unless otherwise defined herein, scientific and technical terms used in conjunction with the present invention shall have the meanings commonly understood by those of ordinary skill in the art. Exemplary methods and materials are described below, but methods and materials similar or equivalent to those described herein can also be used in the practice or testing of the present invention.

As used herein, the term “patient” or “subject” refers to an organism that is to undergo the various tests provided by the technology. The term “subject” includes animals, preferably mammals, including humans. In one or more preferred embodiments, the subject is a human child. In one or more preferred embodiments, the subject is a human child 3 years old or younger.

As used herein, the term “diagnosis” refers to a method that allows a technical person to estimate and even determine whether a subject suffers from a given disease or condition or is likely to develop a given disease or condition in the future. A technical person often makes a diagnosis based on one or more diagnostic indicators, such as one or more metabolites in urine, particularly one or more of the 20 metabolites described in this invention, and particularly one or more of the 5 metabolites described in this invention. The content of one of these metabolites or a combination of multiple content indicates the presence, severity, or absence of autism.

As used herein, the terms “model”, “diagnostic model” and “mathematical model” can be used interchangeably. They refer to the quantitative relationship between things described in mathematical language or formulas used for predicting, especially for diagnosing diseases, for example, the relationship between markers and diseases. It reveals the inherent correlation between the marker and the disease to a certain extent, and it is used as a direct basis for determining the disease during diagnosis. The “model”, “diagnostic model” and “mathematical model” herein may also be the “predetermined algorithm” in the device for diagnosing autism of the present invention.

As used herein, the term “marker” refers to substances that have sufficient correlation with autism to allow them to be used in predictive models of autism. They include, but not limited to, metabolites, organic acids, and alcohols. For example, in some embodiments, markers include phneylactic acid, 3-hydroxy-3-methylglutaric acid, phosphoric acid, fumaric acid, 3-oxoglutaric acid, aconitic acid, N-acetylcysteine, malonic acid, tricarboxylic acid, glycolic acid, creatinine, malic acid, oxalic acid, tartaric acid, pyruvic acid, 4-cresol, carboxycitric acid, 3-hydroxyglutaric acid, 2-hydroxybutyric acid and 2-oxoglutaric acid.

As used herein, the terms “autism spectrum disorder” and “autism” can be used interchangeably. It is a broad definition of autism based on the core symptoms of typical autism. It includes both typical autism and atypical autism, as well as symptoms such as Asperger's syndrome, fringe phenotypes in autism, and suspected autism.

As used herein, the term “machine learning algorithm” is an algorithm used by a computer to simulate or implement human learning behaviors in order to acquire new knowledge or skills and reorganize the existing knowledge structure to continuously improve its own performance. In machine learning, the sample is generally divided into three independent sets, that is, training set, validation set, and testing set. Among them, the training set is used to build the model.

As used herein, the term “eXtreme Gradient Boosting (XGBoost)” is an optimized distributed gradient boosting library, which is characterized by high efficiency, flexibility and portability. It implements machine learning algorithms under the framework of gradient boosting. XGBoost provides parallel tree promotion (also known as GBDT, GBM), which can quickly and accurately solve many data sciences problems. The same codes run on major distributed environments (Hadoop, SGE, MPI) and can solve problems beyond billions of examples.

Among various diseases, researchers have found that metabolic abnormalities related to autism spectrum disorders include phenylketonuria, purine metabolism disorder, folate deficiency in brain development, succinate semialdehyde dehydrogenase deficiency, Smith-Lemli-Opitz syndrome, and so on.

Studies have reported changes in the metabolism of metabolites in patients with autism spectrum disorder. Some metabolites have been confirmed in multiple studies, while some metabolites are only found in a single study. Gas chromatography-mass spectrometry (GC/MS) can be used to evaluate the metabolic disorder of the levels of multiple metabolites in the urine of children with autism spectrum disorder and patients without autism spectrum disorder. In general, the most common metabolic disturbances in children with autism spectrum disorders are microbial metabolites, niacin metabolism, mitochondrial-related metabolites and amino acid metabolites. However, these studies have limitations such as different races, different regions, and very small sample sizes. Therefore, it is necessary to carry out a large sample size of autism spectrum disorder-related metabolic research in the Chinese children.

Considering the complexity of autism spectrum disorder, it is not enough to consider only the rise or fall of a few metabolites. A more comprehensive and accurate diagnosis based on a wider range of metabolites is needed. Therefore, in the field of autism spectrum disorder diagnosis, there is an urgent need for a method of establishing a diagnosis model of autism spectrum disorder and a diagnosis model.

The embodiments of the present invention provide a method for establishing a mathematical model for diagnosing autism spectrum disorder, which includes the following steps:

(1) Recruiting autistic patients, especially children who have been diagnosed with autism, and corresponding healthy individuals;

(2) Sampling from patients and healthy individuals, preferably urine samples. The method of collecting urine can be found in the “Guidelines for the Collection and Processing of Urine Specimens” issued by the Ministry of Health of China. The urine collection process is a non-invasive process to avoid pain caused by other invasive sampling, such as blood sampling;

(3) Detecting metabolites in urine, preferably using gas chromatography or gas chromatography combined with mass spectrometry. The detection of metabolites in urine can utilize any conventional detection methods in the art, such as liquid chromatography, particularly high performance liquid chromatography or a combination of high performance liquid chromatography and mass spectrometry. The advantage of chromatographic mass spectrometry detection is that it can detect multiple metabolites at the same time; and

(4) Using three algorithms (PLSDA, SVM and XGBoost) to process the detected metabolite data and optimize the establishment of a diagnostic model. The calibration and optimization of parameters are key steps in model construction. Among them, the first two algorithms, PLSDA and SVM, have been used in many related studies. This invention applies the XGBoost algorithm to the construction of a urine metabolite model for the first time to distinguish between autistic children and normal development groups. The results show that the autism diagnostic model based on urine metabolites constructed by XGBoost has a very high AUC, reaching above 0.9. Such an efficient detection rate is unique in research and diagnosis related to autism.

In this method, metabolites include but are not limited to Phenylactic acid, 3-Hydroxy-3-Methylglutaric acid, Phosphoric acid, Fumaric acid, 3-Oxoglutaric acid, Aconitic acid, N-Acetylcysteine, Malonic acid, Tricarboxylic acid, Glycolic acid, Creatinine, Malic acid, Oxalic acid, Tartaric acid, Pyruvic acid, 4-Cresol, Carboxycitric acid, 3-Hydroxyglutaric acid, 2-Hydroxybutyric acid, and 2-Oxoglutaric acid. The above 20 metabolites are the top 20 metabolites that contribute the most to autism in the mathematical model constructed by the XGBoost algorithm. The test results prove that the detection rate of the diagnostic model based on these 20 metabolites is very high.

Alternatively, the metabolites include Phenylactic acid, Aconitic acid, Phosphoric acid, 3-Oxoglutaric acid, and Carboxycitric acid. These five metabolites are the top five metabolites that are most significantly related to autism in urine. The metabolites of this species can be used for the diagnosis of autism and the study of the pathogenesis of autism.

The embodiment of the present inventions provides a method for diagnosing autism, including the following steps:

(A) Collecting samples of subjects, especially children with autism, especially urine samples;

(B) Detecting the content of multiple metabolites in the sample, especially using gas chromatography-mass spectrometry; and

(C) Using the autism diagnostic model of the present invention, especially the diagnostic model established by XGBoost to process the measured content of various metabolites.

### Subject Selection and Determination of Metabolites in Samples

The experiments in this invention have been ethically approved by Peking Union Medical College Hospital (#ZS-824). From December 2014 to February 2018, children who suffered from autism (ASD) and children who were in normal developmental stage (TD) were included in the research and evaluated by experienced specialists.

The children in the control group (TD) were primary school students studying in Beijing, and the children with autism (ASD) came from Beijing Herun Clinic. The inclusion criteria were as defined in the fourth edition of the “Diagnostic and Statistical Manual of Mental Disorders” (DSM-4); exclusion criteria include: 1) the presence of other diseases, such as diabetes or phenylketonuria; 2) the presence of certain factors that may interfere with the detection of urine metabolites (such as renal failure, liver insufficiency, dietary intervention therapy, etc.); 3) the diagnosis of other neuropsychiatric diseases; 4) parents cannot assist in completing the assessment.

The urine samples of the research subjects were collected. In order to ensure the quality of the samples, several requirements were strictly complied with throughout the sampling process: the subjects were not allowed to use antibiotics within one month before sampling, were not allowed to take probiotics within 2 weeks, and were not allowed to eat fruits or tomatoes within 24 hours; on the day of sampling, the mid-section urine of the first morning urine was obtained and placed in a sterile tube, and then quickly place the sample on dry ice or in the refrigerator for refrigeration.

All assessments of children's behaviors and eating habits are provided by parents or professional third-party organizations. The form is made strictly in accordance with relevant standards and completed after providing a detailed research introduction and description. This study follows the principle of collecting samples in the home or outpatient environment to ensure that external factors do not affect the samples.

The metabolites in the urine samples were determined by the Great Plains Laboratory by the GS-MS method (gas chromatography-mass spectrometry method).

### Comparison of the Three Algorithms and the Establishment of a Diagnosis Model for Autism

In order to eliminate the influence of the difference in urine concentration between samples, the sample data obtained by the GC/MS method was first standardized by creatinine, and then the data was further processed by scaling and centering. In order to avoid data pollution between model building and model testing process, we reserved an independent testing set from the entire data set and did not expose it to any modeling process. In this way, we minimized the overfitting effect in the testing. The testing set and training set were separated by a random process, and the proportion of samples in the control group and the ASD group remained approximately equal in the two testing sets.

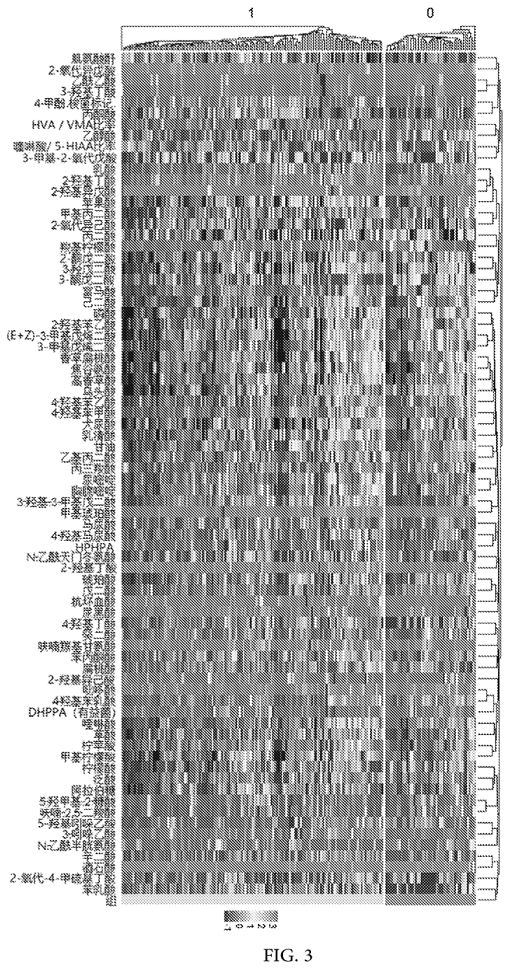

Data analysis used a T test of two independent samples to compare metabolite values between subgroups. The false discovery rate (FDR) method was used for multiple comparisons. The R package “Complex Heatmap” was used to generate a heat map (FIG. 3) to show the possible associations between various metabolites. The heat map has two dimensions, corresponding to the sample and its related metabolic pathways. The identified potential biomarkers have also been marked in the heat map.

In the modeling process, we first used the training set containing 76 metabolites to train the model and adjust the algorithm parameters, and the AUROC was optimized by leaving-one-out cross-validation method. Modeling algorithms include Partial Least Squares Discriminant Analysis (PLS-DA, R mixOmics package), Support Vector Machine (SVM, Re1071 package) and XGBoost (eXtreme Gradient Boosting, R XGBoost package). The generated model based on 76 metabolites (Table 1) was designated as the complete model, and then an independent testing set was used to evaluate the performance of the complete model.

TABLE 1
Metabolites in urine

| I. | Proliferation of Intestinal Microbes |
|---|---|
| A. | Yeast and Fungus Markers |
| 1 | Citricmalic acid |
| 2 | 5-Hydroxymethyl-2-Furoic acid |
| 3 | 3-Oxoglutaric acid |
| 4 | Furan-2,5-dicarboxylic acid |
| 5 | Furan carbonyl glycine |
| 6 | Tartaric acid |
| 7 | Arabinose |
| 8 | Carboxycitric acid |
| 9 | Tricarboxylic acid |
| B. | Absorption Disorders and Bacterial Markers |
| 10 | 2-Hydroxyphenylacetic acid |
| 11 | 4-Hydroxyphenylacetic acid |
| 12 | 4-Hydroxybenzoic acid |
| 13 | 4-Hydroxyhippuric acid |
| 14 | Hippuric acid |
| 15 | 3-indole acetic acid |
| 16 | Succinic acid |
| 17 | HPHPA (Clostridium marker) |
| 18 | 4-Cresol (Clostridium marker) |
| 19 | DHPPA (Probiotics) |
| II. | Oxalate Metabolites |
| 20 | Glycerin |
| 21 | Glycolic acid |
| 22 | Oxalic acid |
| III. | Glycolysis Cycle Metabolites |
| 23 | Lactic acid |
| 24 | Pyruvic acid |
| 25 | 2-Hydroxybutyric acid |
| IV. | Krebs Cycle Metabolites |
| 26 | Fumaric acid |
| 27 | Malic acid |
| 28 | 2-Oxoglutaric acid |
| 29 | Aconitic acid |
| 30 | Citric acid |
| V. | Neurotransmitter Metabolites |
| 31 | Homovanillic acid (HVA) |
| 32 | Vanilla mandelic acid (VMA) |
| 33 | HVA/VMA ratio |
| 34 | 5-Hydroxyindole acetic acid (5-HIAA) |
| 35 | Quinolinic acid |
| 36 | Kynuric acid |
| 37 | Quinolinic acid/5-HIAA ratio |
| VI. | Metabolism of Pyrimidines and Folate |
| 38 | Uracil |
| 39 | Thymine |
| VII. | Oxidative Stress of Ketones and Fatty Acids |
| 40 | 3-Hydroxybutyric acid |
| 41 | Acetoacetic acid |
| 42 | Hydroxybutyrate |
| 43 | Ethylmalonic acid |
| 44 | Methylsuccinic acid |
| 45 | Adipic acid |
| 46 | Suberic acid |
| 47 | Sebacic acid |
| VIII. | Vitamins Markers |
| | Vitamin B12 marker |
| 48 | Methylmalonic acid |
| | Vitamin B6 marker |
| 49 | Pyridoxic acid (B6) |
| | Vitamin B5 marker |
| 50 | Pantothenic acid (B5) |
| | Vitamin B2 (riboflavin) marker |
| 51 | Glutaric acid |
| | Vitamin C marker |
| 52 | Ascorbic acid |
| | Vitamin Q10 (Co-enzyme Q10) marker |
| 53 | 3-Hydroxy-3-Methylglutaric acid |
| | Glutathione Precursor and Chelating |
| | Agent Marker |
| 54 | N-acetylcysteine (NAC) |
| | Vitamin H (Biotin) Marker |
| 55 | Methyl citric acid |
| IX. | Detoxification Reaction Markers |
| 56 | Pyroglutamic acid |
| 57 | Orotic acid |
| 58 | 2-Hydroxybutyric Acid |
| X. | Amino Acid Metabolites |
| 59 | 2-Hydroxyisovaleric acid |
| 60 | 2-Oxoisovaleric acid |
| 61 | 3-Methyl-2-oxopentanoic acid |
| 62 | 2-Hydroxyisohexanoic acid |
| 63 | 2-Oxoisohexanoic acid |
| 64 | 2-Oxo-4-methylthiobutyric acid |
| 65 | Mandelic acid |
| 66 | Phenylactic acid |
| 67 | Phenylpyruvate |
| 68 | Homogentisic acid |
| 69 | 4-Hydroxyphneylactic acid |
| 70 | N-Acetylaspartic acid |
| 71 | Malonic acid |
| 72 | 3-Methylglutaric acid |
| 73 | 3-Hydroxyglutarate |
| 74 | (E + Z)-3-Methylglutaconic acid |
| XI. | Bone Metabolism |
| 75 | Phosphoric acid |
| XII. | Urine Concentration |
| 76 | Creatinine |

Autism group (ASD) and control group (TD) enrolled 156 and 64 subjects, respectively. Males in the ASD group accounted for 80.13%, with a median age of 6 years; males in the control group males accounted for 73.44%, with a median age of 5 years. There was no significant difference in age between the two groups (Table 2).

TABLE 2
Basic characteristics of children with autism and
children in the control group

| | Autism Group | Control Group |
|---|---|---|
| | n = 156 | n = 65 |
| Age (years) | 6 (4, 9.75) * | 5 (4, 7) * |
| Male (%) | 125 (80.13%) | 47 (73.44%) |
| Female (%) | 31 (19.87%) | 17 (26.56%) |

Note:
All values are expressed in numbers (percentage) or median (P25, P75).

In order to identify potential biomarkers of ASD, we used the voting mechanism of three algorithms to generate the 20 most important metabolites in the classification. First, the three algorithms used the R caret package to determine the importance score of each metabolite. Each algorithm ranked the 76 metabolites (Table 1) in descending order according to the importance score. Then, the importance ranking of each metabolite in the three algorithms was integrated, and the metabolite with a lower sum ranking was selected as a potential biomarker. This screened out the top 20 most important metabolites (Table 3).

TABLE 3
Top 20 Potential Metabolic Markers Evaluated by GC/MS
in Urine Samples in Autism Patients and Control Groups

| | | Differentiation | p-value |
|---|---|---|---|
| | | for Autistic | after FDR |
| Number | Metabolites | Sample | adjustment |
| 1 | Phenylactic | ↑ | 0.000 |
| 2 | 3-Hydroxy-3- | ↑ | 0.004 |
| | methylglutaric acid | | |
| 3 | Phosphoric acid | ↓ | 0.001 |
| 4 | Fumaric acid | ↓ | 0.003 |
| 5 | 3-Oxoglutaric | ↓ | 0.001 |
| 6 | Aconitic acid | ↓ | 0.000 |
| 7 | N-Acetylcysteine | ↓ | 0.056 |
| | (NAC) | | |
| 8 | Malonic acid | ↓ | 0.031 |
| 9 | Tricarboxylic acid | ↓ | 0.052 |
| 10 | Glycolic acid | ↓ | 0.140 |
| 11 | Creatinine | ↑ | 0.010 |
| 12 | Malic acid | ↓ | 0.055 |
| 13 | Oxalic acid | ↑ | 0.025 |
| 14 | Tartaric acid | ↓ | 0.046 |
| 15 | Pyruvic acid | ↑ | 0.013 |
| 16 | 4-Cresol | ↑ | 0.030 |
| 17 | Carboxycitric acid | ↓ | 0.001 |
| 18 | 3-Hydroxyglutaric acid | ↓ | 0.071 |
| 19 | 2-Hydroxybutyric acid | ↑ | 0.330 |
| 20 | 2-Oxoglutaric acid | ↓ | 0.408 |

Note:
P values were calculated using Mann-Whitney test.
↑: Compared with the normal control, the level increased;
↓: Compared with the normal control, the level decreased.

Among the 20 metabolites, Phenylactic acid was significantly increased in children with ASD, while the levels of Aconitic acid, Phosphoric acid, 3-Oxoglutaric acid and Carboxycitric acid in children with ASD were significantly reduced (p<0.005). These metabolites participate in a variety of metabolic pathways, including amino acid metabolism, intestinal flora, energy metabolism (Krebs cycle) and bone salt metabolism.

The 20 metabolites related to autism and the 5 metabolites that are more closely related to autism can be used as potential biomarkers for the auxiliary diagnosis of ASD and important markers for discovering the pathogenesis of autism.

We also trained a model with only the first 20 metabolites (reduced_model_20), and used an independent testing set to evaluate the performance of the model. Furthermore, we selected the first 5 metabolites as the stronger biomarkers, and constructed a model (reduced_model_5) using only these 5 metabolites, and evaluated its performance in the same way as reduced_model_20.

Three algorithms were used to evaluate the metabolic levels of urine metabolites in 156 children with ASD and 64 non-autistic children. The total data set was randomly divided into a training set and a testing set. The training set included 124 ASD children and 51 TD children, and the testing set included 32 ASD and 13 TD children. The two data sets have the same proportion of ASD children. The algorithm is trained based on a training set of 175 samples and tested in a reserved testing set containing 45 samples.

We used the training set to train, and used the testing set to test the model based on 20 metabolites, the model based on 5 metabolites and the full model to get the AUROC values. And we compared the effectiveness through the receiver operating characteristic curve (ROC) and area under the curve (AUROC) (Table 4, FIG. 2).

TABLE 4
The Effects of the Three models on Two Data Sets (AUROC)

| AUROC | | | |
|---|---|---|---|
| Training set | | Testing set | |
| Children with | Control group | Children with | Control group |
| autism (n = 124) | children (n = 51) | autism (n = 32) | children (n = 13) |

| | | |
|---|---|---|
| PLS-DA (Ncomp = 2) | | |
| Full model | 0.864 (0.808-0.916) * | 0.863 (0.743-0.966) |
| Model established | 0.859 (0.804-0.918) | 0.911 (0.762-1) |
| with TOP20 | | |
| metabolites | | |
| Model established | 0.807 (0.725-0.883) | 0.863 (0.687-0.978) |
| with TOP5 | | |
| metabolites | | |
| SVM (kernel = ‘linear’) | | |
| Full model | 0.833 (0.758-0.9) | 0.791 (0.634-0.943) |
| Model established | 0.868 (0.798-0.917) | 0.868 (0.714-0.99) |
| with TOP20 | | |
| metabolites | | |
| Model established | 0.763 (0.686-0.824) | 0.805 (0.613-0.938) |
| using TOP5 | | |
| metabolites | | |
| XGBoost | | |
| (max_depth = 2, | | |
| eta = 0.15, | | |
| nrounds = 200) | | |
| Full model | 0.931 (0.889-0.963) | 0.940 (0.834-0.998) |
| Model established | 0.937 (0.9-0.97) | 0.930 (0.831-1) |
| with TOP20 | | |
| metabolites | | |
| Model established | 0.914 (0.869-0.957) | 0.899 (0.774-0.986) |
| using TOP5 | | |
| metabolites | | |

Note:
All figures were expressed in terms of the area under the receiver operating characteristic curve (confidence interval). The confidence interval was estimated by Bootstrapp for 2000 times. The relevant parameters of the algorithm are shown in parentheses after the algorithm.

The results show that the three methods are effective in distinguishing children with autism from children with normal development. During the training process, the AUROC (area under the receiver operating characteristic curve) of the autism diagnostic model based on PLS-DA training was 0.864, and the AUROC of the autism diagnostic model based on SVM training was 0.833, while based on XGBoost, the AUROC of the autism diagnostic model produced by method training is 0.931. XGBoost produced the best results among the three algorithms (AUROC=0.931).

Then, we used the testing set to test the autism diagnostic model trained by the PLS-DA method, the autism diagnostic model trained by the SVM method, and the autism diagnostic model trained by the XGBoost method. The AUROC of the autism diagnostic model generated by the PLS-DA method was 0.863, the AUROC of the autism diagnostic model generated by the SVM method was 0.719, and the AUROC of the autism diagnostic model generated by the XGBoost method was 0.940. Therefore, the autism diagnosis model produced by the XGBoost method is most effective and best suited for diagnosing autism. And the model based on the above 20 metabolites (in Table 3) generated by the XGBoost method has very good AUROC values (0.937 and 0.930 for the training set and testing set, respectively), so it is very suitable for diagnosing autism or predicting the probability of autism.

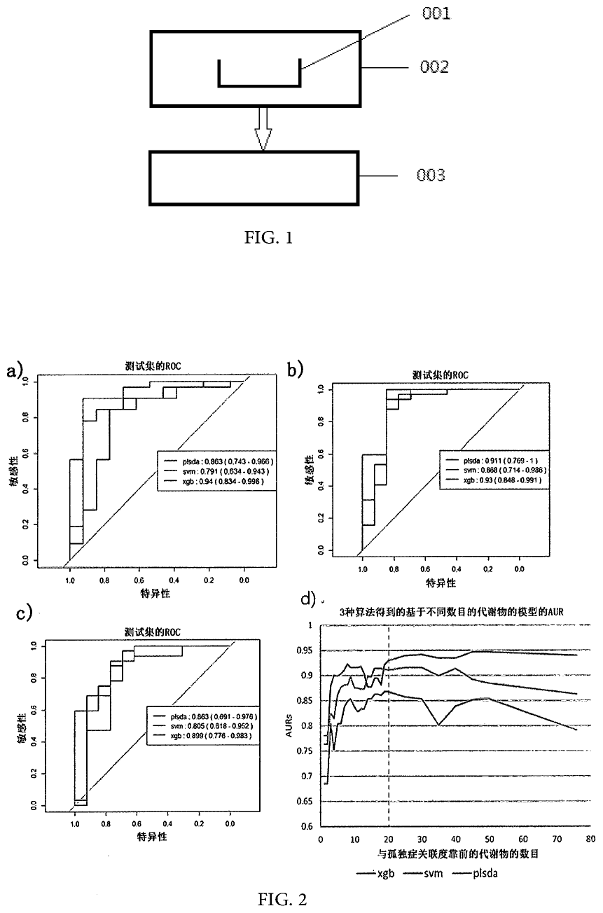

As shown in FIG. 1, according to at least one embodiment of the present invention, a device for diagnosing autism spectrum disorder is provided, which includes an accommodation space 001, a testing unit 002, and a calculation and determination unit 003.

The accommodating space 001 is configured to place a sample of the subject, and the accommodating space 001 is placed so that the sample can be directly or indirectly tested by the testing unit 002.

In one embodiment of the present invention, the sample includes at least one of urine, blood, phlegm, nasopharyngeal secretions, body fluids, or feces. In another embodiment of the present invention, the sample is urine.

Wherein, the markers include at least one of Phenylactic acid, 3-Hydroxy-3-Methylglutaric acid, Phosphoric acid, Fumaric acid, 3-Oxoglutaric acid, Aconitic acid, N-Acetylcysteine, Malonic acid, Tricarboxylic acid, Glycolic acid, Creatinine, Malic acid, Oxalic acid, Tartaric acid, Pyruvic acid, 4-Cresol, Carboxycitric acid, 3-Hydroxyglutaric acid, 2-Hydroxybutyric acid, or 2-Oxoglutaric acid. In other words, the sample may contain at least one of the above-mentioned substances and all combinations of the above-mentioned substances.

In one embodiment of the present invention, the marker includes at least one of Phenylactic acid, Aconitic acid, Phosphoric acid, 3-Oxoglutaric acid, or Carboxycitric acid. In another embodiment of the present invention, the marker is composed of Phenylactic acid, Aconitic acid, Phosphoric acid, 3-Oxoglutaric acid and Carboxycitric acid.

The testing unit 002 is configured to detect the marker of the sample and obtain the content of the marker. In one embodiment, the testing unit 002 adopts a gas chromatography detection method to obtain the content of the marker of the sample. In one embodiment, the testing unit 002 uses a combination of gas chromatography and mass spectrometry to obtain the marker content of the sample.

As shown in the figure, the calculation and determination unit 003 is in communication with the testing unit 002, and obtains the content of the marker of the sample from the testing unit 002. The calculation unit 003 calculates the content of the marker based on a predetermined algorithm to obtain an indication of whether the subject is ill.

For example, in an embodiment of the present invention, the calculation unit 003, based on one of the Partial Least Squares Discrimination Analysis algorithm (PLSDA), Support Vector Machine (SVM), or eXtreme Gradient Boosting algorithm (XGBoost), calculates the marker content of the sample obtained from the detection unit 002 to obtain an indication of whether the subject is ill.

The Partial least squares discriminant analysis is a multivariate statistical analysis method used for discriminant analysis. Discriminant analysis is a common statistical analysis method that determines how to classify research objects based on the values of several observed or measured variables. The principle is to separately train the characteristics of different processed samples (such as observation samples, control samples), generate training sets, and testing the credibility of the training sets.

The Support vector machine is a machine learning algorithm based on statistical learning theory. Its basic idea is to find the two most significant classification lines so that it can correctly divide the two types of data and ensure the maximum classification interval.

The eXtreme gradient boosting algorithm is an optimized distributed gradient boosting library designed to achieve high efficiency, flexibility and portability. It implements machine learning algorithms under the framework of gradient boosting. The eXtreme Gradient Boosting algorithm provides parallel tree boosting (also known as GBDT, GBM), which can quickly and accurately solve many data sciences problems.

The above are only the preferred embodiments of the present invention and are not used to limit the present disclosure. For those skilled in the art, the present disclosure may have various modifications and changes. Any modifications, equivalent replacements, improvements, etc., made within the spirit and principle of the present disclosure shall be included in the protection scope of the present invention.

### INDUSTRIAL APPLICABILITY

The present disclosure applies machine learning algorithms to disease marker screening and disease diagnosis mathematical model establishment. Specifically, partial least square discriminant analysis, support vector machine and XGBoost algorithm were used to screen out the 20 most weighted markers, and a highly effective diagnostic model was established using XGBoost.

The present disclosure uses urine as a sample. The urine collection method is simple and easy to implement, and the urine collection is a non-invasive process, and highly operable in the clinic. These are beneficial to the diagnosis of autistic patients.

The present disclosure has successfully established a diagnosis model for autism based on 20 or more metabolites. And using the mathematical model of the present disclosure to process the sample parameters, the specificity, sensitivity, and practicability of diagnosis are greatly improved.

Chromatography-mass spectrometry can quickly detect 20 or more metabolites at once. This method is fast and relatively inexpensive.

The mathematical model and device of the present disclosure can be used for the early diagnosis of autism spectrum disorder. It overcomes the bottleneck in autism spectrum disorder disease, i.e., diagnosing without objective indicators. It solves the technical problem to diagnose children with autism aged 3 years or younger and under.

A comprehensive study of the metabolites of patients with autism spectrum disorder will also provide clues for the study of the biological phenotype and disease pathogenesis of autism spectrum disorder.

1-25. (canceled)

26. A device for diagnosing autism spectrum disorder, including
an accommodating space configured to place a sample of a subject;
a testing unit configured to test a marker of the sample to obtain the content of the marker; and
a calculation and determination unit configured to calculate the content of the marker according to a predetermined algorithm to obtain an indication of whether the subject suffers from autism spectrum disorder;
wherein the marker comprises Phenylactic acid, Aconitic acid, Phosphoric acid, 3-Oxoglutaric acid and Carboxycitric acid;
the predetermined algorithm is XGBoost.

27. The device according to claim 26, wherein the marker comprises Phenylactic acid, 3-Hydroxy-3-Methylglutaric acid, Phosphoric acid, Fumaric acid, 3-Oxoglutaric acid, Aconitic acid, N-Acetylcysteine, Malonic acid, Tricarboxylic acid, Glycolic acid, Creatinine, Malic acid, Oxalic acid, Tartaric acid, Pyruvic acid, 4-Cresol, Carboxycitric acid, 3-Hydroxyglutaric acid, 2-Hydroxybutyric acid, and 2-Oxoglutaric acid.

28. The device according to claim 26, wherein the testing unit comprising a gas chromatography detection device and a mass spectrometry detection device.

29. The device according to claim 26, the sample comprises at least one of urine, blood, sputum, nasopharyngeal secretions, body fluids, or feces.

30. The device according to claim 26, wherein the autism spectrum disorder includes Rett syndrome, childhood disintegration, Asperger's syndrome, or unspecified generalized developmental disorder.

31. The device according to claim 26, wherein the subject is a human.

32. The device according to claim 31, wherein the subject is a child.

33. The device according to claim 27, wherein the testing unit comprising a gas chromatography detection device and a mass spectrometry detection device.

34. The device according to claim 27, the sample comprises at least one of urine, blood, sputum, nasopharyngeal secretions, body fluids, or feces.

35. The device according to claim 27, wherein the autism spectrum disorder includes Rett syndrome, childhood disintegration, Asperger's syndrome, or unspecified generalized developmental disorder.

36. The device according to claim 27, wherein the subject is a human.

37. The device according to claim 31, wherein the subject is a child.
